(19) United States
(12) Patent Application Publication
(10) Pub. No.: US 2009/0181990 A1
(43) Pub. Date: Jul. 16, 2009

(54) STABLE AMORPHOUS FORM OF PEMETREXED DISODIUM

(76) Inventors: Nileshkumar S. Patel, Gujarat (IN); Srinivasu Kilaru, Gujarat (IN); Rajamannar Thennati, Gujarat (IN)

Correspondence Address: SCHWEGMAN, LUNDBERG & WOESSNER, P.A., P.O. BOX 2938, MINNEAPOLIS, MN, 55402, US

(21) Appl. No.: 12/341,888

(22) Filed: Dec. 22, 2008

(30) Foreign Application Priority Data
Dec. 23, 2007 (IN) 2313/MUM/2007

(51) Int. Cl. A61K 31/519; C07D 487/04; A61P 35/00; C12N 5/08; A61P 17/06

(52) U.S. Cl. 514/265.1; 544/280; 435/375

(57) ABSTRACT

The present invention provides an amorphous form of pemetrexed disodium, a process for preparing the amorphous form of pemetrexed disodium, and a pharmaceutical composition that includes the amorphous form of pemetrexed disodium and a pharmaceutically acceptable excipient. The process for preparing the amorphous form of pemetrexed disodium includes dissolving pemetrexed disodium in a solvent, to obtain a solution, optionally filtering the solution, and recovering the amorphous form from the solution. The present invention provides for the use of the amorphous form of pemetrexed disodium in medical therapy and treatment.

### RELATED APPLICATION

This application claims the benefit of priority, under 35 U.S.C. Section 119, to Indian Patent Application Serial No. 2313/MUM/2007 filed on Dec. 23, 2007, which is incorporated herein by reference in its entirety.

### FIELD OF THE INVENTION

The present invention relates to a stable amorphous form of pemetrexed disodium of formula (I):

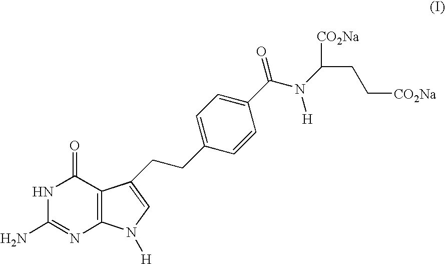

### BACKGROUND OF THE INVENTION

Pemetrexed disodium is a multitargeted antifolate agent approved as a single agent for the treatment of non-small cell lung cancer, and in combination with cisplatin for the treatment of patients with malignant pleural mesothelioma, under the trade name Alimta®.

Pemetrexed disodium is available in a number of crystalline forms. Barnett et al, Organic Process Research & Development, 1999, 3, 184-188 discloses synthesis and crystallization of pemetrexed disodium from water-ethanol. The product obtained by the process disclosed herein is the 2.5 hydrate of pemetrexed disodium.

U.S. Pat. No. 7,138,521 discloses a crystalline heptahydrate form of pemetrexed disodium, which has enhanced stability when compared to the known 2.5 hydrate.

To date, workers have concentrated on producing stable crystalline forms of pemetrexed disodium and it is believed that there has been no disclosure of any non-crystalline form of this active.

### SUMMARY OF THE INVENTION

We have found a new form of pemetrexed disodium, which is an amorphous form, as characterized by powder X-ray diffraction. Surprisingly, we have found that it is possible to prepare an amorphous form of pemetrexed disodium, and that this form is relatively stable. The amorphous form is relatively stable, contrary to expectations. The amorphous form is relatively stable, as it retains it's amorphous character under a variety of storage conditions. The amorphous form is particularly advantageously characterized by a bulk density in the range of 0.15 to 0.35 gm/ml.

The present invention provides an amorphous form of pemetrexed disodium.

The present invention also provides for the compound of formula (I):

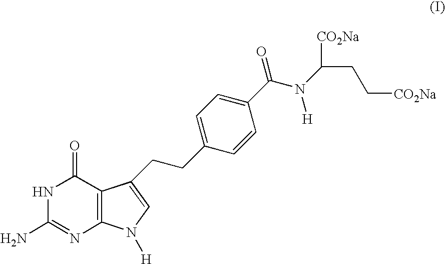

The present invention also provides a process for preparing the amorphous form of pemetrexed disodium that includes dissolving pemetrexed disodium in a solvent, to obtain a solution, optionally filtering the solution and recovering the amorphous form from the solution.

The present invention also provides a pharmaceutical composition that includes the amorphous form of pemetrexed disodium and pharmaceutically acceptable excipients.

The present invention also provides a method of treating non-small cell lung cancer in a mammal. The method includes administering to a mammal in need of such treatment an effective amount of the amorphous form of pemetrexed disodium.

The present invention also provides a method of treating malignant pleural mesothelioma in a mammal. The method includes administering to a mammal in need of such treatment an effective amount of the amorphous form of pemetrexed disodium, in combination with an effective amount of cisplatin.

The present invention also provides a method of inhibiting the growth of a neoplasm. The method includes contacting the neoplasm with an amount of an amorphous form of pemetrexed disodium, effective to inhibit the growth.

The neoplasm may be choriocarcinoma, leukemia, adenocarcinoma of the female breast, epidermid cancers of the head and neck, squamous or small-cell lung cancer, lymphosarcomas, or any combination thereof.

The contacting can be in vivo or in vitro.

The present invention also provides a method of treating mycosis fungoides or psoriasis in a mammal. The method includes administering to a mammal in need of such treatment, an effective amount of an amorphous form of pemetrexed disodium.

### DETAILED DESCRIPTION OF THE INVENTION

The present invention provides an amorphous form of pemetrexed disodium.

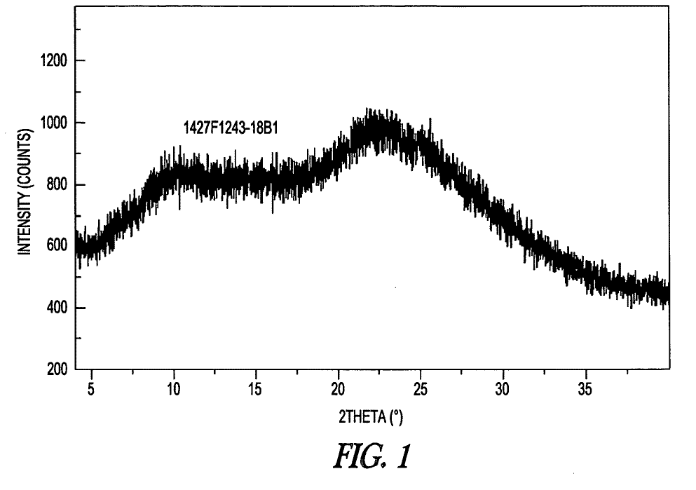

In one embodiment, the amorphous pemetrexed disodium is characterized by the X-ray diffraction pattern of FIG. 1.

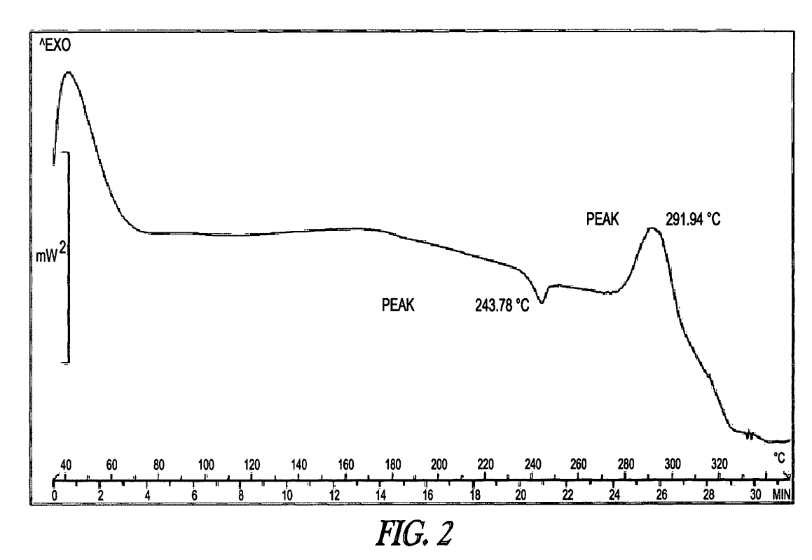

In another embodiment, the amorphous pemetrexed disodium is characterized by differential scanning calorimetry ((DSC) profile (FIG. 2).

In another embodiment, the amorphous pemetrexed disodium is characterized by peaks in the Raman spectrum at about 3064, 2921, 1611, 1534, 1438, 1343, 1293, 1190, 1157, 1076, 1010, 903, 872, 820, 639, 533, 373, 103 cm⁻¹. The tolerance of the measurements is ±2 cm⁻¹. Table 1 in Example 1 also depicts the Raman spectra of amorphous form of the present invention.

In a further embodiment, the amorphous form of pemetrexed disodium is also characterized by the Raman Spectra substantially shown in FIG. 3.

The amorphous pemetrexed can be characterized by any of the above mentioned methods individually, or in any combination of these individual methods.

In one embodiment, the amorphous form of pemetrexed disodium, as described herein, has a water content ranging from about 5% to about 21%.

In one embodiment, the amorphous form of pemetrexed disodium as described herein is relatively stable, as it retains its amorphous nature and lacks significant impurity formation when stored under varying temperature and humidity conditions.

The expression “stable” or “relatively stable” when used in conjunction with amorphous pemetrexed disodium means that, within the scope of sound pharmacological judgment, the material can be stored without significant chemical degradation or transformation of amorphous form. The expression includes pemetrexed disodium that can be stored under a suitable atmosphere without transformation, or without significant transformation, of its amorphous form and without more than 5% degradation over a period of at least 28 days, and more preferably over a period of at least 3 months, and most preferably over a period of at least 6 months. Preferably, the degradation is less than 2%, and more preferably less than 1%. Percentage degradation may be determined by HPLC as described herein. In embodiments, the suitable atmosphere is an inert gas, e.g. nitrogen. In embodiments, the storage temperature is not more than 40° C., e.g. not more than 25° C. Included are embodiments in which the storage conditions include relative humidity not exceeding 80%, e.g. not exceeding 60%. One suitable storage condition is 25° C./60% relative humidity.

The term “stable” or “relatively stable,” as used herein, thus implies that the pemetrexed disodium described herein maintains its amorphous form and lacks significant formation of impurities, while being stored as described herein.

In one embodiment, the amorphous form of pemetrexed disodium as described herein remains stable when subjected to the following real time and accelerated conditions wherein the amorphous form of pemetrexed disodium was stored:
at 2-8° C. for a period of six months
25° C./60% relative humidity for a period of six months
   in that the amorphous form is retained as indicated by XRD and there was no significant formation of impurities as measured by HPLC when the samples were analyzed after a period of six months.

The samples of amorphous form of pemetrexed disodium were stored in double seal poly bags followed by aluminum bag sealed under nitrogen with oxygen trapper.

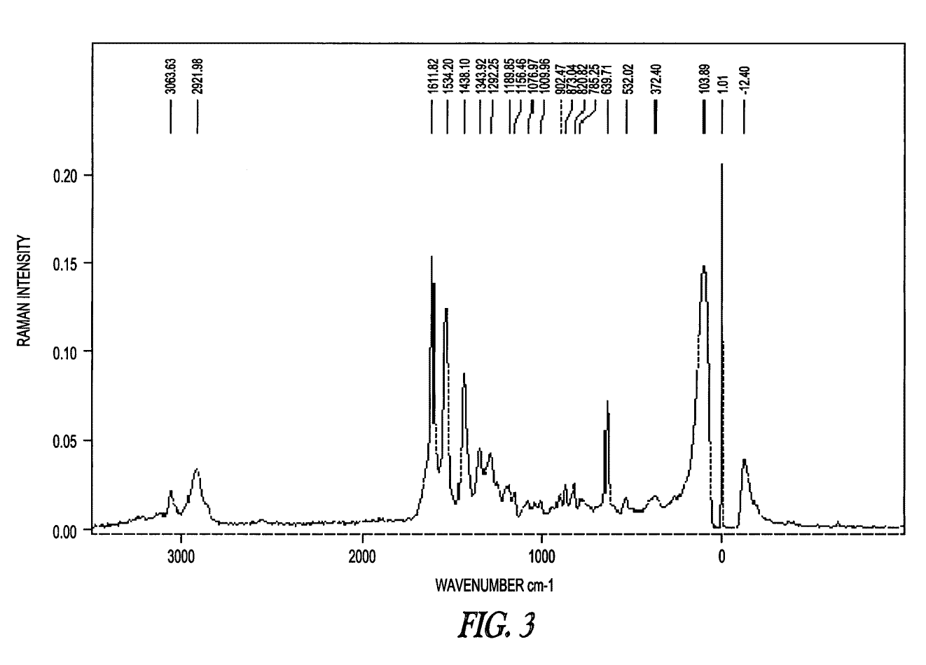

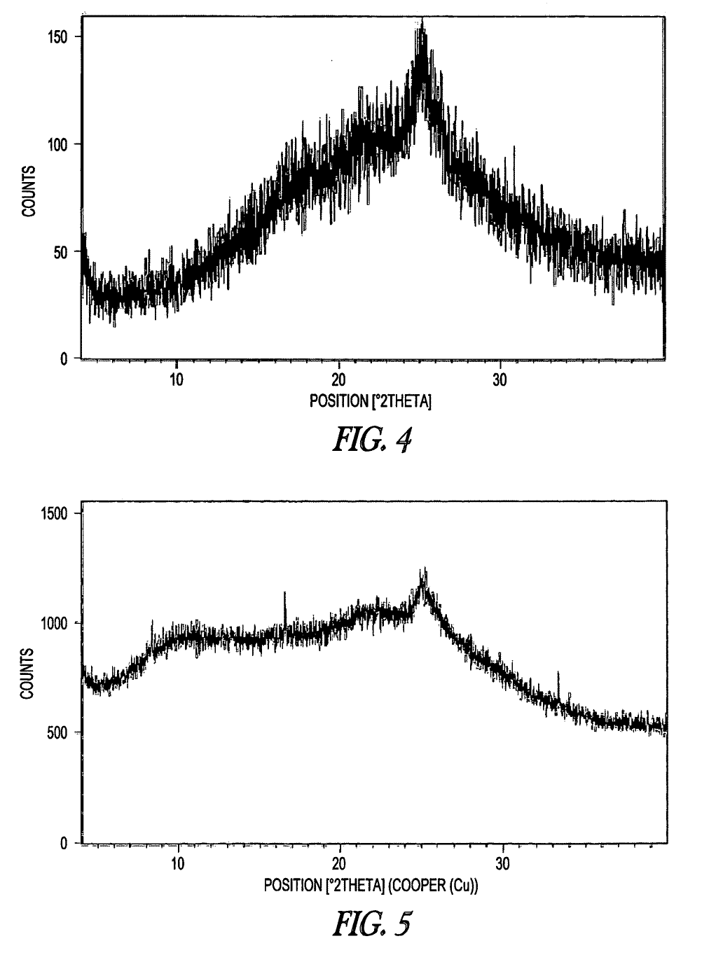

In one embodiment, the amorphous form of pemetrexed disodium (XRD as depicted in FIG. 4) has an initial moisture content of about 9.0%, and HPLC purity of 99.68% as determined by standard analytical procedures known in the art. In another embodiment, the amorphous form of pemetrexed disodium (XRD as depicted in FIG. 5) has an initial moisture content of about 16.0%, and HPLC purity of 99.66% as determined by standard analytical procedures known in the art. Example 5 discloses that the above amorphous form of pemetrexed disodium of the present invention is stable when stored under accelerated conditions as it does not transform to any other form as indicated by the powder X-ray diffractograms (XRD) (FIGS. 6-10) which clearly indicate that the amorphous form is conserved and there is no significant formation of impurity as indicated by the HPLC purity values after storing under these conditions for a period of six months

Examples 5 and 6 also disclose the moisture content variation in the samples of amorphous pemetrexed disodium, as described herein, upon storage under various conditions Thus depending on the storage conditions, the packaging material and the initial moisture content, the moisture content in the samples on storage may vary from 5-21%; however under all circumstances the amorphous form was conserved and there was no significant formation of impurities as indicated by HPLC values despite varying the moisture content indicating that the amorphous form of pemetrexed disodium, as described herein, is stable.

In one embodiment, the amorphous form of pemetrexed disodium, as described herein, exhibits bulk density in the range of 0.15 to 0.35 gm/ml.

In one embodiment, the amorphous form of pemetrexed disodium, as described herein, exhibits tapped density in the range of 0.25 to 0.45 gm/ml.

As referred to herein the term “bulk density” is the weight of the sample divided by its non packed volume and the term “tapped density” is the weight of the sample divided by its packed volume. The units of bulk density are grams (gm) per cubic centimeter (cc) or grams (gm) per milliliter (ml). A powder having low bulk density will be lightweight, fluffy and have greater surface area. A powder with high density will be much more compact and dense, exist as harder particles and will result in a more flowable product compared to powder with low bulk density.

In a preferred embodiment, the amorphous form of pemetrexed disodium, as described herein, exhibits a bulk density in the range of 0.21 to 0.24 gm/ml, and preferably a bulk density of 0.226 gm/ml±0.01 gm/ml.

In another preferred embodiment, the amorphous form of pemetrexed disodium, as described herein, exhibits a tapped density in the range 0.35 to 0.39 gm/ml, and preferably a bulk density of 0.369 gm/ml±0.01 gm/ml.

The present invention also relates to a process for the preparation of an amorphous form of pemetrexed disodium.

In one embodiment, the present invention provides a process for preparing the amorphous form of pemetrexed disodium comprising the steps of:
a) dissolving pemetrexed disodium in a solvent to obtain a solution;
b) optionally filtering the solution; and
c) recovering the amorphous form of pemetrexed disodium from the solution.

The term “solvent,” as used herein, refers to any solvent or a solvent mixture in which pemetrexed disodium is soluble. The solvent may be selected from water, one or more alkanols, or a combination thereof. The alkanol may be a C₁₋₁₀ alkanol, e.g., methanol, ethanol, isopropanol and the like.

Thus in one embodiment, the solvent is water. In an alternative embodiment, the solvent is a mixture of water and one or more alkanols.

The solution of pemetrexed disodium in solvent can optionally be filtered.

The amorphous form of pemetrexed disodium, as described herein, may then be obtained by drying the solution of pemetrexed disodium in solvent. Conventional processes such as freeze drying, spray drying, flash drying and the like may be used for recovering the amorphous form pemetrexed disodium from the solution of pemetrexed disodium. Preferably the amorphous form is a freeze-dried or a spray-dried product. Most preferably, it is a freeze-dried product.

In one preferred embodiment, the solvent for dissolving pemetrexed disodium is water.

In one embodiment, pemetrexed disodium is dissolved in water to obtain a clear solution. This solution is then freeze dried to obtain the amorphous form of the pemetrexed disodium.

In one embodiment, about 5 to 15 volumes of water are used for dissolving the pemetrexed disodium. In a preferred embodiment, about 8 to 12 volumes of water are used.

In one embodiment, the pH of the pemetrexed disodium solution in water prior to freeze drying is in the range of 6.5 to 7.5. In a preferred embodiment, the pH of the pemetrexed disodium solution in water prior to freeze drying is about 6.95.

The freeze drying is carried out at the temperature in the range of about −40 to about +25° C., and under vacuum in the range of 0.1 mm to 10 mm. The freeze drying is carried out for a period of about 15 to about 60 hours. Based on the time of freeze drying and batch size, the water content of the amorphous form of the present invention can be varied in the range of about 5-21%.

In one instance when the freeze drying is carried out for 28 hours, an amorphous form of pemetrexed disodium with a water content of about 7.46% is obtained. In another instance when the freeze drying is carried out for about 53 hours, an amorphous form of pemetrexed disodium with a water content of about 5.82% is obtained. For larger batch sizes depending on the desired water content, the freeze drying time may be increased.

The pemetrexed disodium used for preparing the amorphous form of pemetrexed disodium of the instant invention may be prepared by any process known in the art. Preferably the starting pemetrexed disodium is prepared by dissolving the pemetrexed diacid in a solution of sodium hydroxide and water. The pemetrexed disodium is then precipitated by adding ethanol. The precipitated pemetrexed disodium is then isolated and used for preparing the amorphous form.

The amorphous pemetrexed disodium may be administered alone but will generally be administered in admixture with a pharmaceutically acceptable diluent or carrier selected with regard to the intended route of administration and standard pharmaceutical practice. The amorphous form of pemetrexed disodium of the present invention may be formulated into conventional dosage forms such as, for example, tablets, pills, suspensions, emulsions, granules, capsules, injection preparations suitable for reconstitution and the like.

For example, it may be administered orally in the form of tablets containing such excipients as starch or lactose, or in capsules either alone or in admixture with excipients, or in the form of elixirs or suspensions containing flavouring or colouring agents. It may be injected parenterally, for example, intravenously, intramuscularly or subcutaneously.

For parenteral administration, it is best used in the form of a sterile aqueous solution which may contain other solutes, for example, enough salts or glucose to make the solution isotonic with blood.

The amorphous pemetrexed disodium may be administered as capsule formulations. Such formulations may be prepared by mixing the amorphous material together with suitable carriers or excipients such as microcrystalline cellulose, dried maize starch, colloidal silicon dioxide and magnesium stearate.

The amorphous form of pemetrexed disodium of the present invention may be used alone or in combination with one or more other therapeutically active agents.

It will be appreciated that reference to medical treatment means curative, palliative or prophylactic treatment. In specific embodiments, reference to medical treatment means curative treatment.

The present invention also relates to the use of amorphous pemetrexed disodium for treating cancer, and in particular for treating non-small cell lung cancer as described below.

In particular, the amorphous form can be used, under the supervision of qualified professionals, to inhibit the growth of neoplasms including choriocarcinoma, leukemia, adenocarcinoma of the female breast, epidermid cancers of the head and neck, squamous or small-cell lung cancer, and various lymphosarcomas. It can also be used to treat mycosis fungoides and psoriasis.

The compounds preferably are administered parenterally, alone or in combination with other therapeutic agents including other anti-neoplastic agents, steroids, etc., to a mammal in need of medical treatment. Parenteral routes of administration include intramuscular, intrathecal, intravenous and intra-arterial. Dosage regimens must be titrated to the particular neoplasm, the condition of the patient, and the response but generally doses will be from about 10 to about 100 mg/day for 5-10 days or single daily administration of 250-500 mg, repeated periodically; e.g. every 14 days. The term mammal includes a human patient.

The present invention is further illustrated by the following examples which are provided merely to be exemplary of the invention and are not intended to limit the scope of the invention.

### EXAMPLES

### Experimental Methods

General description of the equipment.

X-ray diffraction data were acquired using a PANalyticalX′pert PRO X-ray diffractometer model.

System description: K_{α1}=1.54060 A⁰, voltage 45 kV, current 40 mA, Xray source: Cu.

Experiment parameters: pattern measured between 2θ=4° and 2θ=40° with 0.05° increments; count time was 0.5 second per increment.

DSC was performed using Metler Toledo DSC822^{e}.

Freeze drying was performed on a freeze dryer by Martinchrist alpha 1-4 LD plus model.

Bulk and tapped densities were measured using Electrolab ETD-1020 tap density tester (USP).

The water content measurements were carried out using a METTLER TOLEDO Model: DL31Karl Fischer Titrator according to standard procedures.

Purity by HPLC was performed by Waters 2690 Seperations module.system.

Raman spectra was recorded on—FT-Raman spectrophotometer, Make-Bruker Optics, Switzerland, Model-RFS 100/S

Laser—750 mW Nd: YAG laser operating at 1064 nm.

Detector-Liquid nitrogen cooled Germanium detector.

No. of scans-50 (Scanning ranges from 3500 cm-1 to −999 cm-1)

Laser power—350 mW.

### Example 1

TABLE 1
Raman spectra of 4 batches of amotrphous
pemetrexed of the present invention

| | | | |
|---|---|---|---|
| 3066 | 3066 | 3063 | 3065 |
| 2925 | 2919 | 2921 | 2920 |
| 1611 | 1611 | 1611 | 1611 |
| 1535 | 1534 | 1534 | 1534 |
| 1438 | 1438 | 1438 | 1438 |
| 1348 | 1343 | 1343 | 1343 |
| 1292 | 1294 | 1292 | 1292 |
| 1189 | 1191 | 1189 | 1191 |
| 1155 | 1158 | 1156 | 1157 |
| 1078 | 1075 | 1076 | 1076 |
| 1008 | 1011 | 1009 | 1012 |
| 905 | 903 | 902 | 904 |
| 872 | 872 | 873 | 871 |
| 821 | 821 | 820 | 820 |
| 639 | 640 | 639 | 639 |
| 534 | 534 | 532 | 533 |
| 367 | 371 | 372 | 375 |
| 104 | 103 | 103 | 103 |

### Example 2

5.0 gm Pemetrexed disodium is dissolved in 50 ml demineralised water at 40-45° C. and filtered to obtain a particle-free filtrate. The filtrate is then freeze dried at about −20° C. to 0° C., at about 5 mm vacuum, using CHRiST freeze-drier for 28 hours to obtain 5.25 g of amorphous pemetrexed disodium.

X-ray diffraction data as indicated in FIG. 1.

Water content: 7.46%

HPLC purity 99.66%

Bulk density: 0.226 gm/ml

Tapped density: 0.369 gm/ml

### Example 3

13.2 gm Pemetrexed disodium was dissolved in 80 ml demineralised water at 40-45° C. 20 ml of additional demineralised water was added and the solution is then freeze dried at about −20 to 0° C., at about 1-2 mm vacuum, using CHRiST freeze-drier for 20 hours to obtain 10.5 g of amorphous pemetrexed disodium with an initial moisture content of 11.38% to 12.02%.

This material was dried under high vacuum till moisture content was 9%.

X-ray diffraction data as indicated in FIG. 4 indicates amorphous form is retained.

Water content: 9%

HPLC purity 99.68%

The material was kept outside at room temperature for 15 hrs.

X-ray diffraction data as indicated in FIG. 5 indicates amorphous form is retained.

Water content: 16%

HPLC purity 99.66%

### Example 4

Solution of 0.074 Kg of Sodium hydroxide in 0.4 L Water for Injection is added to the suspension of 0.4 Kg Pemetrexed diacid in 1.2 L Water for Injection below 25° C. Obtained slight suspension is heated at 40-45° C. and filtered. 6.0 L of Abs. Ethanol is added to the filtrate below 30° C. and resulting precipitated product is stirred for 1.0 hr, filtered, washed with 0.8 L Abs. Ethanol and suck dried for 30-60 min. Above isolated wet material is dissolved in 3.2 L Water for Injection at 40° C., filtered and washed with 0.8 L Water for Injection. Resulting solution is freeze dried in Virtis freeze drier, Model No.: Genius 25XL, Sr No.: 214706 for 87 hrs (12 hrs for freezing+76 hrs for drying) at −40° C. to 0° C. using vacuum.

Yield=398 gm

water content 6.82%

HPLC purity 99.61%

### Example 5

### Amorphous Pemetrexed Disodium Stability Study

This example demonstrates the stability of amorphous form of pemetrexed obtained in example 2 with initial moisture content of 9% and 16%.

The samples were packed in double seal poly bags followed by Aluminium bag sealed under nitrogen with oxygen trapper and stored
at 2-8° C. for a period of six months
25° C./60% relative humidity (RH) for a period of six months.

The purity of the sample was checked by HPLC and XRD was recorded for the samples stored under these conditions after 2 months and 6 months.

I. Amorphous Form of Pemetrexed Disodium, with Moisture Content of 9% Prior to Storage

| | | |
|---|---|---|
| HPLC | % Moisture Content | XRD |

(a) Condition: 25° C. RH 60%

| | | | |
|---|---|---|---|
| Initial | 99.68 | 9.04% | Amorphous |
| 2 Months | 99.72 | 8.39% | |
| 6 Months | 99.67 | 8.85% | Amorphous |
| | | | (FIG. 6) |

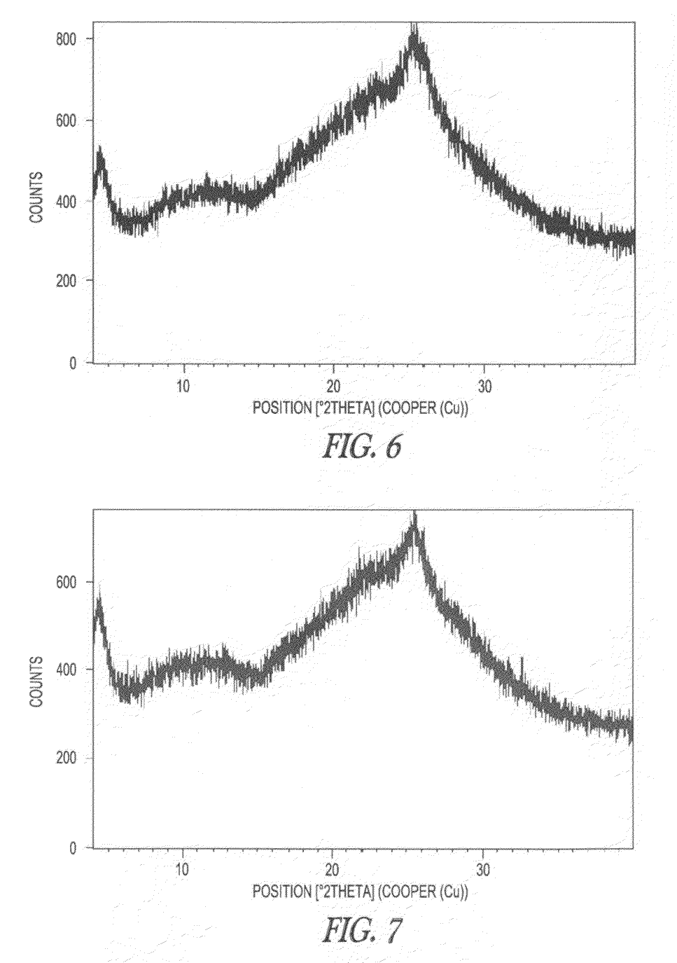

(b) Condition: 2-8° C.

| | | | |
|---|---|---|---|
| Initial | 99.68 | 9.04% | Amorphous |
| 2 Months | 99.72 | 8.96% | |
| 6 Months | 99.62 | 9.39% | Amorphous |
| | | | (FIG. 7) |

II. Amorphous Form of Pemetrexed Disodium, with Moisture Content of 16% Prior to Storage

| | | |
|---|---|---|
| HPLC | % MC | XRD |

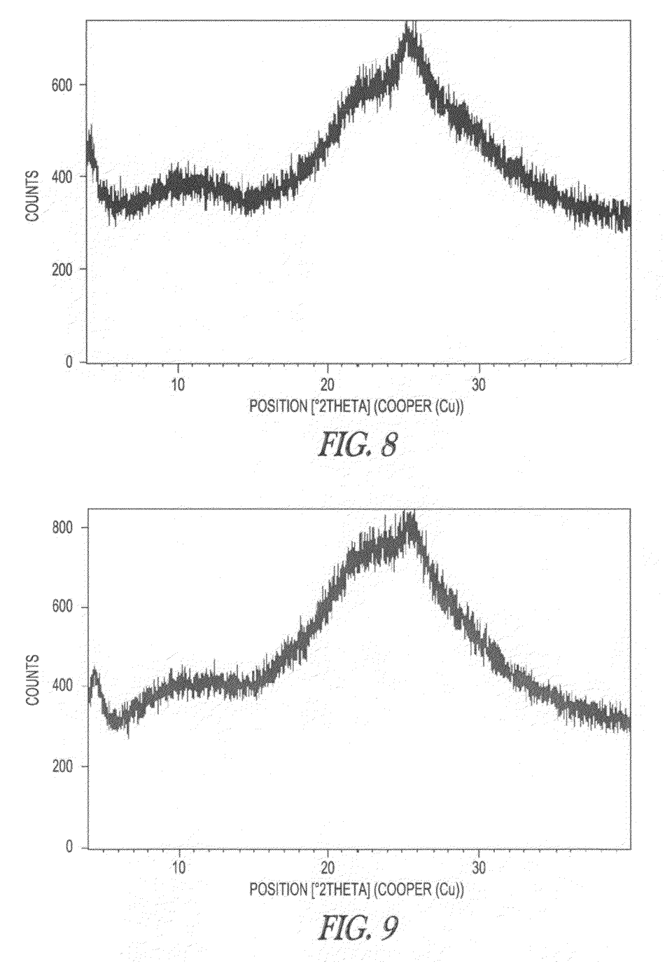

a. Condition: 25° C. RH 60%

| | | | |
|---|---|---|---|
| Initial | 99.66 | 15.92% | Amorphous |
| 2 Months | 99.72 | 10.26% | |
| 6 Months | 99.63 | 11.45% | Amorphous |
| | | | (FIG. 8) |

b. Condition: 2-8° C.

| | | | |
|---|---|---|---|
| Initial | 99.66 | 15.92% | Amorphous |
| 2 Months | 99.72 | 11.68% | |
| 6 Months | 99.59 | 12.31% | Amorphous |
| | | | (FIG. 9) |

The above results indicate that all the samples tested conserved the amorphous form after being stored at the specified storage period under specific conditions. Further more there was no significant change in the initial chemical purity after storage.

### Example 6

This example demonstrates that amorphous form is conserved under various storage conditions.

| | | | % | % |
|---|---|---|---|---|
| | | | Moisture | Moisture |
| | | | Content | Content |
| Storage conditions | Packing conditions | XRD | Initial | after storage |
| 40° C./75% relative | packed in double polythene | Amorphous form | 5.48% | 18.27% |
| humidity for 7 days | bags and further kept in a | conserved | | |
| | fiber drum | | | |
| 80° C. for 7 days | open petriplate | Amorphous form | 5.48% | 6.24% |
| | | conserved | | |
| Room temperature | open petriplate | Amorphous form | 5.48% | 20.03% |
| for 24 hours | | conserved | | |
| Room temperature | double polythene bags | Amorphous form | 5.48% | 18.56 |
| for 40 days | | conserved | | |

### Example 7

HPLC Analysis method

Reagent: Water: milliQ,

Sodium perchlorate: AR Grade

Perchloric acid: AR Grade

Acetonitrile: J. T. Baker gradient

Trifluroacetic acid: AR Grade

Buffer solution: 6.1 g of sodium perchlorate into a 1000 ml water. Adjust the pH to 3.0 (+0.1) with perchloric acid.

### Mobile Phase A:

mixture of buffer and acetonitrile in the proportion of (90:10).

### Mobile Phase B:

mixture of buffer and acetonitrile in the proportion of (10:90).

Diluent—1: mixture of water and acetonitrile in the ratio of 50:50.

Diluent—2: mixture of water and acetonitrile in the ratio of 90:10.

### Standard Stock Solution:

Transfer accurately weighed 1.5 mg impurity-E RS and into a 200 ml volumetric flask. Dissolve in and dilute upto mark with diluent-1.

### Blank Solution

Add 10 ml diluent-2 and 5011 of 3% trifluro acetic acid to a 50 ml volumetric flask, and dilute upto mark with diluent-2.

### System Suitability Solution

Transfer about 25 mg accurately weighed pemetrexed disodium sample in to a 50 ml volumetric flask. First add 10 ml of diluent-2 and sonicate to dissolve the contents. Then add 50 μl of 3% trifluro acetic acid (prepared in water) and add 5 ml of standard stock solution and dilute up to mark with diluent-2.

### Sample Preparation

Transfer about 25 mg accurately weighed pemetrexed disodium sample in to a 50 ml volumetric flask. First add 10 ml of diluent-2 and sonicate to dissolve the contents. Then add 50 μl of 3% trifluro acetic acid (prepared in water) and dilute up to mark with diluent-2 (500 μg/ml).

### Chromatographic System:

Use a suitable high pressure liquid chromatography system equipped with

Column: 250 mm×4.6 mm containing 5μ packing material (suggested column—Inertsil ODS 3V)

Detector: UV detector set to 240 nm

Cooler temp: 5° C.

Flow rate: about 1.5 ml/min.

The system is also equipped to deliver the two phases in a programmed manner as shown in the following table:

### Gradient Programme:

| | | |
|---|---|---|
| 0 | 92 | 8 |
| 15 | 85 | 15 |
| 30 | 65 | 35 |
| 35 | 65 | 35 |
| 36 | 92 | 8 |
| 40 | 92 | 8 |

### Procedure:

Inject 20 μl of blank and system suitability solution into the chromatograph set to above conditions and record the chromatograms up to 40 min.

Calculate the resolution between pemetrexed disodium and impurity-E. The resolution should not be less than 3.0. Calculate the Number of theoretical plate and tailing factor for pemetrexed peak. Number of theoretical plate is NLT 4000 and tailing factor is NMT 2.0.

Inject 20 μl of test solution and calculate the chromatographic purity by area normalisation method.

1. An amorphous form of pemetrexed disodium.

2. A compound of formula (I):

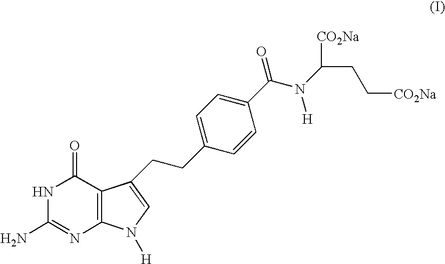

3. Amorphous pemetrexed disodium characterized by peaks in Raman spectrum at about 3064, 2921, 1611, 1534, 1438, 1343, 1293, 1190, 1157, 1076, 1010, 903, 872, 820, 639, 533, 373, 103 cm⁻¹.

4. A process for preparing an amorphous form of pemetrexed disodium comprising:
a. dissolving pemetrexed disodium in a solvent to obtain a solution;
b. optionally filtering the solution; and
c. recovering the amorphous form of pemetrexed disodium from the solution.

5. The process of claim 3, wherein the amorphous form of pemetrexed disodium is recovered from the solution by freeze drying, spray drying, flash drying or any combination thereof.

6. The process of claim 3, wherein the solvent is selected from the group consisting of water, an alkanol, or a mixture thereof.

7. A pharmaceutical composition comprising an amorphous form of pemetrexed disodium, and a pharmaceutically acceptable excipient.

8. A method of treating non-small cell lung cancer in a mammal, the method comprising administering to a mammal in need of such treatment an effective amount of an amorphous form of pemetrexed disodium.

9. The method of claim 8, wherein the mammal is a human.

10. The method of claim 8, wherein the amorphous form of pemetrexed disodium is a compound of formula (I):

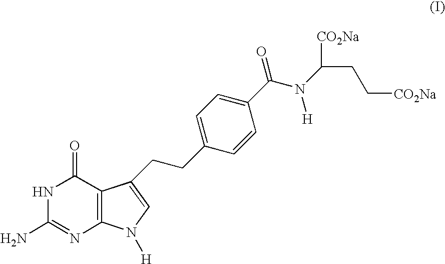

11. A method of treating malignant pleural mesothelioma in a mammal, the method comprising administering to a mammal in need of such treatment an effective amount of an amorphous form of pemetrexed disodium, in combination with an effective amount of cisplatin.

12. The method of claim 11, wherein the mammal is a human.

13. The method of claim 11, wherein the amorphous form of pemetrexed disodium is a compound of formula (I):

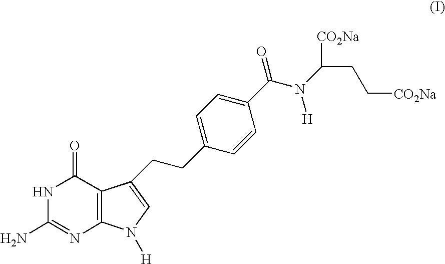

14. A method of inhibiting the growth of a neoplasm, the method comprising contacting the neoplasm with an amount of an amorphous form of pemetrexed disodium, effective to inhibit the growth.

15. The method of claim 14, wherein the neoplasm is choriocarcinoma, leukemia, adenocarcinoma of the female breast, epidermid cancers of the head and neck, squamous or small-cell lung cancer, lymphosarcomas, or any combination thereof.

16. The method of claim 14, wherein the contacting is in vivo.

17. The method of claim 14, wherein the contacting is in vitro.

18. The method of claim 14, wherein the amorphous form of pemetrexed disodium is a compound of formula (I):

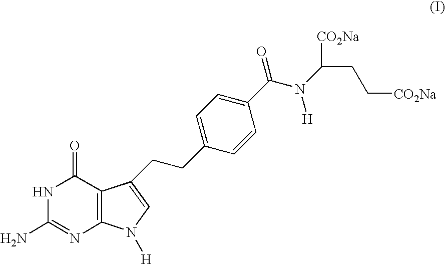

19. A method of treating mycosis fungoides or psoriasis in a mammal, the method comprising administering to a mammal in need of such treatment, an effective amount of an amorphous form of pemetrexed disodium.

20. The method of claim 19, wherein the mammal is a human.

21. The method of claim 19, wherein the amorphous form of pemetrexed disodium is a compound of formula (I): [chemical structure]
